(19) United States
(12) Patent Application Publication
(10) Pub. No.: US 2021/0147844 A1
(43) Pub. Date: May 20, 2021

(54) Method for Site-Specific Mutagenesis of Medicago Sativa Genes by Using CRISPR/Cas9 System

(71) Applicant: Guangdong Sanjie Forage Biotechnology Co., Ltd, Guangzhou (CN)

(72) Inventors: Haitao Chen, Guangzhou (CN); Wen Wang, Guangzhou (CN); Xiongping Xie, Guangzhou (CN); Qiang Qiu, Guangzhou (CN); Zhanhuan Shang, Guangzhou (CN); Kexian Su, Guangzhou (CN); Hui He, Guangzhou (CN)

(73) Assignee: Guangdong Sanjie Forage Biotechnology Co., Ltd, Guangzhou (CN)

(21) Appl. No.: 17/141,354

(22) Filed: Jan. 5, 2021

Related U.S. Application Data
(63) Continuation of application No. 17/257,617.

(30) Foreign Application Priority Data
Jul. 4, 2018 (CN) 201810724589.8

(51) Int. Cl. C12N 15/113; C12N 9/22

(52) U.S. Cl. CPC C12N 15/113; C12N 2310/20; C12N 9/22

(57) ABSTRACT

A method for site-specific mutagenesis of Medicago sativa genes by using a CRISPR/Cas9 system. The method comprises: first constructing a binary expression vector MsCRISPR/Cas9 that can be used for transforming Medicago sativa by Agrobacterium tumefaciens; then designing a target site for a target gene, and ligating the DNA fragment containing the guide sequence of the target site into MsCRISPR/Cas9 to construct a vector MsCRISPR/Cas9::target; and then transforming the Medicago sativa by Agrobacterium tumefaciens, and generating, by screening, a mutant transformed plant with the target gene mutated. According to the method, an MtU6 promoter is used for driving sgRNA transcription in the Medicago sativa.

### TECHNICAL FIELD

The present disclosure belongs to the technical field of genome editing, and specifically pertains to a method for site-specific mutagenesis of Medicago sativa genes by using a CRISPR/Cas9 system.

### BACKGROUND

Medicago sativa is a kind of legume forage cultivated widely, being rich in high quality dietary fibres, edible proteins, various vitamins (including vitamin B, vitamin C, vitamin E, etc.), various beneficial minerals as well as saponin, flavonoids, carotenoids, phenolic acid and other bioactive components, high in protein content and having good palatability, which is of great significance for improving the feed level of livestocks. In addition to being forage crops, the well-developed root system of Medicago sativa plays an important role in preventing and controlling soil erosion as well as environmental management. In addition, because the roots of Medicago sativa are symbiotic with a large number of nitrogen-fixing microorganisms, so Medicago sativa also has important significance in improving the fertility of soil. Researches on gene functions of Medicago sativa are helpful to improve the agronomic traits of Medicago sativa. Using gene silencing technologies to suppress the expression of target genes in Medicago sativa is a very efficient means to study gene functions and cultivate new varieties.

Although spontaneous mutations often occur in nature, their frequency is extremely low. The mutation rate of spontaneous mutations at most loci in each generation is about 10⁻⁵ or 10⁻⁶, and only mutations that occur in gametes can be passed on to the next generation. Ideal mutants can be created in a short time through treating plant materials by chemical mutagenesis, physical mutagenesis as well as DNA insertional mutagenesis. The frequency of mutations caused by mutagens is high, which is hundreds or even thousands of times higher than the frequency of spontaneous mutations. Moreover, mutagens-induced mutations have a wide range of variations and various types, sometimes may induce new mutations that are rare or have not occurred in nature. However, mutations introduced by mutagens are also random, and it is very difficult to generate null mutants for a specific site. Similarly, mutations induced by mutagenesis can be passed on to the next generation only if they occur in gametes. Targeted gene silencing is an effective method to study specific gene functions. At present, several genome editing techniques such as RNAi (RNA interference), as well as ZFN, TALLEN, CRISPR/Cas9 have been developed for targeted gene silencing. The RNAi technology mainly degrades mRNA sequences of the target genes and down-regulate the translation level of mRNA, thus inhibiting the expression of target genes. However, when the expression of target genes is reduced by the RNAi technology, the gene sequence of the target genes has not been mutated. In contrast, gene silencing by means of ZFN, TALLEN, CRISPR/Cas9 technologies makes the gene sequence mutated at the DNA level, resulting in complete loss of gene function. Compared with RNAi, the three technologies are more stable and efficient. When using TALEN and ZFN technologies to silence the target genes, the amino acid sequence elements that recognize different bases are assembled according to the target site, wherein each target site has its own unique protein sequence, thus resulting in a time-consuming, labor-intensive and costly process. Different from TALLEN and ZFN, the CRISPR/Cas9 technology identify the target site through the guide sequence on sgRNA. The guide sequence recognizes the target site by complementary base pairing, which is more convenient, cheap and easy to operate. Therefore, since the CRISPR/Cas9 technology was firstly applied in genome editing in 2013, it has developed rapidly and extensively. The CRISPR/Cas9 system is an acquired immune system of Streptococcus, which can resist the invasion of foreign genes. It is composed of CRISPR and specific Cas9 protein. CRISPR (Clustered Regularly Interspaced Short Palindromic Repeats) is a new type of DNA repeating sequence, which consists of a series of short highly conserved forward repeating sequences and spaced sequences arranged alternately. Cas9 protein is a kind of multidomain protein consisting of 1,409 amino acids, which contains two nuclease domains, RuvC-like and HNH. The CRISPR/Cas9-based genome editing system includes 2 parts: sgRNA and enzyme Cas9. Such a system can specifically identify the target site adjacent to PAM (NGG) motif, and cut it at 3 nt upstream of PAM to form a DSB (double strains break), after then the organism utilizes its own repairing mechanism, such as homologous recombination (HR) and non-homologous end joining (NHEJ), to repair the damaged DNA. The repairing process of NHEJ can produce Indel (including deletions and insertions), which further leads to frameshift mutation of the DNA coding sequence.

Medicago sativa is a strictly cross-pollinated autotetraploid plant, with four alleles at each locus. For a particular locus, the most ideal way to generate null mutant strains with stable inheritance is creating enough mutants with four alleles simultaneously mutated. Null mutant strains can also be generated by repeatedly hybridizing the natural or mutagenic induced heterozygous mutants to accumulate the mutations, but this is a time-consuming, labor-intensive, high input, and inefficient process. Therefore, in conclusion, cultivating novel alfalfa varieties by recurrent hybridization and selection is particularly difficult, especially for recessive gene mutations. It is of great significance for the researching and breeding of Medicago sativa to develop an efficient targeted mutation technology that can be used to create enough null mutants within one generation. Currently, it is known to employ the RNAi technology to reduce the gene expression level of Medicago sativa, and there have been no reports of mutants created by using genome editing ZFN, TALEN, and CRISPR/Cas9. As described above, compared with the genome editing technologies such as ZFN, TALEN, and CRISPR/Cas9, RNAi restricts its own application due to incomplete silencing and instability. Null mutants with stable inheritance have been successfully generated in rice, arabidopsis, tomatoes, polar, white polar, apples, wheat, corn, soybean and other species through the CRISPR/Cas9 technology, but there have been no reports of genome editing by using CRISPR/Cas9 in Medicago sativa.

### SUMMARY

In order to use genome editing technology to modify the target genes in the autotetraploid plant Medicago sativa, and apply it to production practice, we developed a method for site-specific mutagenesis by using a CRISPR/Cas9 system, thus achieving precise site-directed gene mutations in Medicago sativa with a mutation rate up to 52%, and generating null mutant plants with ideal phenotype and four mutated alleles (mutation rate reaches 12%). This method takes the lead in establishing an efficient targeted genome editing system in Medicago sativa and has a great significance in the researching and breeding of Medicago sativa. Therefore, the present disclosure is intended to provide a method for site-specific mutagenesis of Medicago sativa genes by using a CRISPR/Cas9 system.

The technical solution of the present disclosure is as below:

A method for site-specific mutagenesis of Medicago sativa genes by using a CRISPR/Cas9 system, wherein, the method comprises the following steps:

Step (1) Constructing a universal binary expression vector MsCRISPR/Cas9 that can be used for transforming Medicago sativa by Agrobacterium tumefaciens;

Step (2) Designing a CRISPR/Cas9-based sgRNA target site for a target gene and ligating the DNA fragment containing the guide sequence of the target site into the universal vector MsCRISPR/Cas9 to construct a vector MsCRISPR/Cas9::target;

Step (3) Transforming the Medicago sativa by the Agrobacterium tumefaciens, and introducing site-specific mutations into the target gene.

Further, the Medicago sativa materials comprise wild varieties, local varieties, bred varieties, and introduced varieties.

Further, the target gene comprises any biologically functional genes or DNA sequences in the Medicago sativa genome.

Further, the complete sequence of the binary vector MsCRISPR/Cas9 for expressing the CRISPR/Cas9 system in the Medicago sativa is as shown in SEQ ID NO. 1.

The technology relies on the transformation of Medicago sativa by Agrobacterium tumefaciens, during which a T-DNA sequence containing a selection marker Hpt gene expression frame, a sgRNA expression frame and a Cas9 gene expression frame is integrated into the Medicago sativa genome through transformation to generate transgene plants, wherein the transformed exogenous expression elements are expressed in Medicago sativa and thus function to generate plants with the gene mutated. An expression vector MsCRISPR/Cas9 is constructed for the transformation by Agrobacterium.

Preferably, the backbone vector of the binary expression vector MsCRISPR/Cas9 for expressing the CRISPR/Cas9 system in the Medicago sativa is a pCambia1300 vector; the pCambia1300 backbone vector contains a T-DNA region that is used to transform the plants; the T-DNA region comprises a left boundary repeat (LB repeat), a right boundary repeat (RB repeat) as well as a sequence between the left and right boundary sequences used for transformation; the sequence between the left and right boundary sequences of the T-DNA region used for transformation comprises a Hpt gene expression frame, a Cas9 gene expression frame, an sgRNA expression frame; the Hpt gene expression frame comprises a CaMV 35S promoter (Enhanced) for driving the transcription of the Hpt gene, a CDS sequence of the Hpt gene and a CaMV poly A terminator for terminating the transcription of the Hpt gene; the Cas9 gene expression frame comprises a 2×CaMV 35S promoter sequence for driving the transcription of the Cas9 gene, a CDS sequence of the Cas9 gene and a Nos terminator sequence for terminating the transcription of Cas9 gene; the sgRNA expression frame comprises a MtU6 promoter from Medicago truncatula for driving the transcription of the sgRNA and a DNA sequence for expressing sgRNA.

In this vector (FIG. 1), LB T-DNA repeat/RB T-DNA repeat represents the left and right boundary repeats of T-DNA; CaMV 35S promoter (Enhanced)/MtU6 promoter/2×CaMV 35S promoter represent promoter elements; Hpt/sgRNA/Cas9 represent gene sequences; CaMV poly (A)/NosT represent terminator sequences; Aarl represents the insertion site of sgRNA guide sequence. As shown in FIG. 1: the Medicago truncatula MtU6 promoter is used to drive the expression of sgRNA; the 2×CaMV 35S promoter is used to drive the expression of Cas9 gene; and the CaMV 35S promoter (Enhanced) is used to drive the expression of the selective marker gene Hpt.

Further, the sequence of the MtU6 promoter is as shown in SEQ ID NO. 2.

Preferably, the DNA sequence for expressing sgRNA is as shown in SEQ ID NO. 3; and the sequence from positions 1 to 30 in the DNA sequence for expressing sgRNA comprises two Aarl restriction endonuclease recognition sites, which are used to construct the expression vector of site-specific mutagenesis for the target gene.

Further, the expression vector MsCRISPR/Cas9::target is constructed as below:

Step (1) Designing a pair of oligonucleotide primers (18-24 bp) according to the target site, which can form DNA fragments with cohesive ends at both ends as below, and synthesize:

5′-T T T G N₁₆₋₂₃-3′
3′-C N₁₆₋₂₃C A A A-5′;

Step (2) Annealing the above synthesized primer pair to form a complementary double-stranded DNA fragment with cohesive ends;

Step (3) Digesting the binary expression vector MsCRISPR/Cas9 with Aarl restriction endonuclease; dephosphorylating through reaction with CIP and then separating by electrophoresis in 0.8-1.2% of agarose gel, purifying the linear vector from gel;

Step (4) Ligating the complementary double-stranded DNA fragment with cohesive ends to the purified MsCRISPR/Cas9 linear vector by using a T4 DNA ligase in a water-bath pot at 16° C. for 10-16 h;

Step (5) Transforming Escherichia coli DH5α competent cells with the ligated products by heat shock, coating on a LB plate medium containing 50 mg/L of kanamycin, and cultivating in an incubator at 37° C. for 12-16 h;

Step (6) Selecting monoclonal bacterials and cultivating them in 1.5 mL LB liquid medium containing 50 mg/L of kanamycin, and culturing in a shaker at 37° C. for 12-16 h;

Step (7) Sequecing the vector of monoclonal bacterials by using a sequencing primer MtU6-T-F, selecting proper monoclonal bacterials and cultivating them in 50-100 mL LB liquid medium containing 50 mg/L of kanamycin, culturing in a shaker at 37° C. for 12-16 h, and extracting the vectors.

Further, the sequence of the sequencing primer MtU6-T-F is as shown in SEQ ID NO. 4.

Preferably, the Medicago sativa is transformed by using Agrobacterium tumefaciens to generate mutant plants through the following steps:

Step (1) Recovery of the strains of Agrobacterium tumefaciens: The Agrobacterium strains after being transformed by heat shock are coated in a YM solid medium (Sigma Co.), and cultured at 25-29° C. for 24 h-48 h; monoclones are selected, inoculated in 30-50 mL YM liquid medium, and cultivated on a shaker at 100-220 r/min at 25-29° C. for 24-48 h; the YM solid medium and the YM liquid medium both comprise 30-60 mg/L of kanamycin and 200-450 mg/L of rifampicin;

Step (2) Acquisition of Medicago sativa callus: The Medicago sativa callus is generated as below:

Plump and well-colored Medicago sativa seeds are selected, soaked with 65-80% alcohol for 1-3 minutes, washed with sterile water for 2-4 times, 0.5-2 minutes for each time; then soaked with 0.05-0.15% mercuric chloride and shaken by hands for 6-12 minutes, and washed with sterile water for 3-5 times; then inoculated on a MS solid medium, and germinated in light in an incubator or in a culture room for 7-16 days;

Cotyledons and hypocotyls of the germinated seedlings are cut into small pieces and inoculated in a callus induction medium, and cultured in dark in an incubator or in a culture room at 25±1° C. for 2-4 days; The ingredients of the callus induction medium are: SH basal medium+1.0-3.0 mg/L 2,4-D (2,4-Difluorophenoxy acetic acid)+0.1-0.3 mg/L KT (kinetin)+0.1-0.5 mg/L casein hydrolysate+15-40 g/L sucrose+5.8-9 g/L agar; Preferably, the ingredients of the callus induction medium are: SH basal medium+2 mg/L 2,4-D+0.2 mg/L KT+0.3 mg/L casein hydrolysate+30 g/L sucrose+8 g/L agar;

Step (3) Transformation of Medicago sativa callus with Agrobacterium tumefaciens: the Medicago sativa callus is transformed with Agrobacterium strains as below: the Agrobacterium tumefaciens strains recovered in step (1) are inoculated in 50-100 mL YM liquid medium and cultured at 25-29° C. on a shaker at 100-220 r/min until the OD value 260/280 is between 0.5-0.8; the bacterial solution is transferred into a 50 mL sterile centrifuge tube, and centrifuged at 0-8° C. in a centrifuge at 3000-4500 r/min for 10-15 minutes; the supernatant is discarded, and a resuspension solution (the MS basal medium) is added for resuspension until the OD value 260/280 is between 0.5-0.8; acetosyringoneat 50-150 μmol/L is added; the callus induced in step (2) is collected into a sterile triangular flask with a breathable and plastic sealing membrane, into which is poured the resuspended Agrobacterium bacterial solution, sealed with its own sealing membrane, and evacuated in a vacuum pump to 0.5 kpa for totally 0.5-1.5 h, during which the flask is shaken gently every 10-15 min; the triangular flask is taken out and shaken at 25-29° C. on a shaker at 100-150 r/min for 0.5-1.5 h; all the bacterial solution is poured out, and the materials are spread out in a sterile culture dish containing filter papers to dry out;

Step (4) Co-cultivation of the Medicago sativa callus with Agrobacterium tumefaciens: The co-cultivation process is as below: The dried transformed materials are inoculated on a co-cultivation medium (spreading a piece of sterilized filter paper in the medium), and cultivated in dark in an incubator at 25±1° C. for 2-5 days; the ingredients of the co-cultivation medium are: MS basal medium+1-3 mg/L 2,4-D+0.1-0.3 mg/L KT+15-40 g/L sucrose+5.8-9 g/L agar+50-150 μmol/L acetosyringone; Preferably, the ingredients of the co-cultivation medium are: MS basal medium+2 mg/L 2,4-D+0.2 mg/L KT+30 g/L sucrose+8 g/L agar+100 μmol/L acetosyringone;

Step (5) Screening cultivation of Medicago sativa callus: The process of screening cultivation is as below: The co-cultivated materials are inoculated on a screening medium, and cultivated in light in an incubator or in a culture room at 25±1° C. for 30-60 days; the ingredients of the screening medium are: SH basal medium+1-3 mg/L 2,4-D+0.1-0.3 mg/L KT+15-40 g/L sucrose+5.8-9 g/L agar+150-450 mg/L cefotaxime+150-450 mg/L carbenicillin+10-50 mg/L hygromycin; Preferably, the ingredients of the screening medium are: SH basal medium+2 mg/L 2,4-D+0.2 mg/L KT+30 g/L sucrose+8 g/L agar+250 mg/L cefotaxime+250 mg/L carbenicillin+15 mg/L hygromycin;

Step (6) Differentiation cultivation of Medicago sativa callus: The materials generated after screening cultivation are transferred onto a differentiation medium, and cultivated in light in an incubator or in a culture room at 25±1° C. for 15-30 days; the ingredients of the differentiation medium are: UM basal medium+0.5-5 g/L casein hydrolysate+0.1-2 mg/L kinetin+15-40 g/L sucrose+5.8-9 g/L agar+150-450 mg/L cefotaxime+4-10 mg/L hygromycin; Preferably, the ingredients of the differentiation medium are: UM basal medium+2 g/L casein hydrolysate+0.4 mg/L kinetin+30 g/L sucrose+8 g/L agar+250 mg/L cefotaxime+5 mg/L hygromycin;

Step (7) Rooting cultivation of regenerated sprouts of Medicago sativa: The differentiated sprouts of 1-3 cm are transferred onto a rooting medium; the ingredients of the medium are: MS basal medium+0.5-2 mg/L indolebutyric acid+15-40 g/L sucrose+5.8-9 g/L agar+150-450 mg/L cefotaxime; Preferably, the ingredients of the rooting medium are: MS basal medium+1 mg/L indolebutyric acid+30 g/L sucrose+8 g/L agar+250 mg/L cefotaxime;

Step (8) Screening and genotyping of Medicago sativa mutants: The methods for screening and genotyping the Medicago sativa mutants comprise PCR-RE, T7E1 enzyme digestion genotyping and targeted deep sequencing. The genotyping method is specifically as below:

PCR-RE genotyping: DNA samples are extracted from the regenerated plants generated above, and specific primers are designed for the amplification of the target gene sequence. In the fragments generated from amplification, if there are restriction endonuclease recognition sites on the cutting sites of Cas9 protein, the mutated sequence cannot be digested by the restriction endonuclease due to the loss of recognition sites. Specific operations are: a. amplifying the fragments flanking the target sites of regenerated plants and control plants by using the specific primers; b. purifying the target fragments generated from amplification, and digesting the target fragments with target-specific restriction endonuclease; c. genotyping the digested products by agarose gel electrophoresis, and the results of electrophoresis are analyzed: after the amplification products of the regenerated seedlings are digested with restriction endonuclease, if there are bands in the electrophoretic bands of the same size as those in the control sequence without enzymatic digestion and there are no other bands, while the control sequence for restriction endonuclease digestion is cut into two smaller bands, it indicates that all alleles of the target gene are mutated and the regenerated seedlings are null mutants; after the amplification products of the regenerated seedlings are digested with restriction endonuclease, if there are bands in the electrophoresis results of the same size as those in the control sequence without enzymatic digestion, and if there are bands of the same size as those in the two smaller bands of the control sequence after digestion with restriction endonuclease, it indicates that only partial alleles of the target genes are mutated and wild-type alleles still exist; after the amplification products of the regenerated seedlings are digested with restriction endonuclease, if there are no bands in the electrophoretic bands of the same size as those in the control sequence without enzymatic digestion, and the bands after enzymatic digestion have the same size as those in the two smaller bands of the control sequence after digestion with restriction endonuclease, it indicates that no allele of the target genes is mutated; d. purifying the above bands which have not been cut, ligating them to pMD19-T vector (Takara Co.) through TA cloning, transforming Escherichia coli competent cells and then selecting a batch of monoclones for sequencing, and analyzing the types of mutation on the target.

T7E1 genotyping: DNA samples are extracted from the regenerated plants generated above, and specific primers are designed for the amplification of the target gene sequence. In the target gene fragments generated from amplification, if there are no restriction endonuclease recognition sites on the cutting sites of Cas9 protein, the mutated plants are genotyped by T7E1 enzymatic digestion. Specific operations are: a. designing specific primers to amplify the sequence flanking the target sites; b. mixing the amplification products of wild-type and regenerated plants; c. denaturing and renaturing the mixed products; d. digesting the renatured products by using T7E1 enzyme (NEB Co.), and genotyping by agarose gel electrophoresis. Analysis of results: if there are mutations in the target genes of regenerated plants, after denaturation and renaturation, they will form a heteroduplex with the amplification products of wild-type plants, and the heteroduplex can be digested into two bands of ideal size by T7E1 enzyme; e. ligating the PCR products of regenerated seedlings which can be digested by T7E1 enzyme to a pMD19-T vector by TA cloning, transforming Escherichia coli competent cells and then selecting a batch of monoclones for sequencing, and analyzing the types of mutation on the target.

Genotyping by targeted sequencing: DNA samples are extracted from the regenerated plants generated above, and amplified the target site with specific amplification primers. PCR products are ligated into a pMD19-T vector by TA cloning, Escherichia coli competent cells is transformed and then a batch of monoclones are selected for targeted sequencing to analyze whether there are mutations on the target as well as the types of mutations.

At the same time, the present disclosure also claims an application of the method for site-specific mutagenesis of Medicago sativa genes by using a CRISPR/Cas9 system in Medicago sativa breeding.

The present disclosure has successfully established a system of CRISPR/Cas9-based genome editing technology in Medicago sativa for the first time, through which the target gene can be successfully mutated and null mutants with obvious phenotype can be generated. To realize genome editing in the autotetraploid plant Medicago sativa, on the basis of the existing platform of generating transgenic plants by transforming Medicago sativa with Agrobacterium tumefaciens, the present disclosure employs an Agrobacterium transforming method to integrate a T-DNA region of an expression frame containing the Hpt gene expression frame, an sgRNA expression frame and a Cas9 gene expression frame into the Medicago sativa genome, wherein these elements are transcribed and translated in Medicago sativa cells and perform their function to generate mutants. The key point of expressing the CRISPR/Cas9 system in Medicago sativa is to find proper promoters, wherein the CaMV 35S promoter (Enhanced) for driving the transcription of Hpt gene and the 2×CaMV 35S promoter for driving the transcription of Cas9 gene have been shown to drive the transcription of transgenes in Medicago sativa; the promoter for driving the transcription of sgRNA is generally U6 or U3 promoter, but there have been no report about the U6/U3 promoter in Medicago sativa. For driving the transcription of sgRNA efficiently, the MtU6 promoter from Medicago truncatula is selected.

Compared with the existing technology for downregulating the gene expression in Medicago sativa by means of RNAi, the present disclosure has the following benefits:

The present disclosure employs the CRISPR/Cas9 technology in the cross-pollinated autotetraploid plant Medicago sativa for the first time, and null mutants are generated; the MtU6 promoter is firstly used in Medicago sativa for driving the transcription of sgRNA.

1. Through the present disclosure, null mutants can be generated within one generation in the cross-pollinated autotetraploid Medicago sativa, which has great value for accelerating the breeding and researching of Medicago sativa.

2. The present disclosure firstly establishes a CRISPR/Cas9 genome editing platform in Medicago sativa based on transformation by Agrobacterium tumefaciens, through which the target genes can be mutated directly, thus making the technology more complete and stable.

3. Compared with natural mutations and mutagens-induced mutations, the present disclosure introduces mutations with higher mutation efficiency, the mutation rate being up to 52% (wherein, the mutation rate of null mutants reaches 12%).

4. The technology opens up a new field for genome editing in Medicago sativa, laying a technical foundation for the subsequent simultaneous editing of multiple genes, large fragment deletion of chromosome, and precise insertion of exogenous gene; at the same time, Cas9 protein can be mutated and inactivated to be dCas9/nCas9 that can be coupled to protein domains with other functions to achieve the expression upregulation/downregulation, methylation/demethylation, and base substitution of the target gene.

### BRIEF DESCRIPTION OF THE DRAWINGS

FIG. 1 is the structure diagram of MsCRISPR/Cas9 expression vector;

FIG. 2 shows the wild and mutant plants of PDS gene generated by transforming the Medicago sativa with MsCRISPR/Cas9::PDS vector which contains the target of PDS gene;

FIG. 3 shows the sequencing results of PCR products of PDS gene of wild and mutant Medicago sativa plants;

FIG. 4 shows the results of PCR-RE analyses for identifying mutants of PALM1 gene;

FIG. 5 shows the leaf morphologies of wild-type and mutant-type plants of the PALM1 gene;

FIG. 6 shows the sequencing results of PCR products of PALM1 gene of wild and mutant Medicago sativa plants.

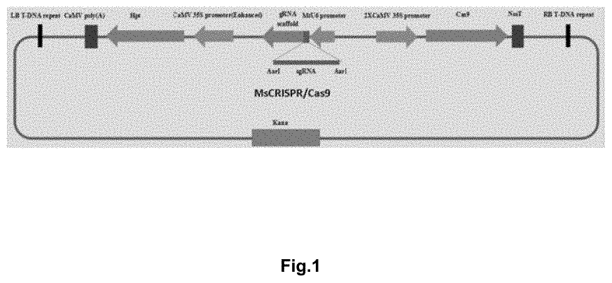

### DETAILED DESCRIPTION

The technical solution of the present disclosure will be further described below in detail in combination with specific examples, but the present disclosure is not limited to the following technical solution.

### Example 1 Mutation of Phytoene Desaturase Encoding Gene PDS in Medicago Sativa by Means of a CRISPR/Cas9 System

Phytoene desaturase is a major rate-limiting enzyme in synthesis of carotenoid, which can catalyze colorless C40 phytonee to generate ξ-carotene, neurosporene, lycopene, 3, 4-dehydrolycopene, 3, 4, 3′4′-dehydrolycopene or 3, 4-dehydroneurosporene, etc. The mutant plants of this gene show albino phenotype, which is easy to observe. Using this gene as the target gene in plants is a convenient method to determine whether the gene knockout system works as well as to evaluate the working efficiency.

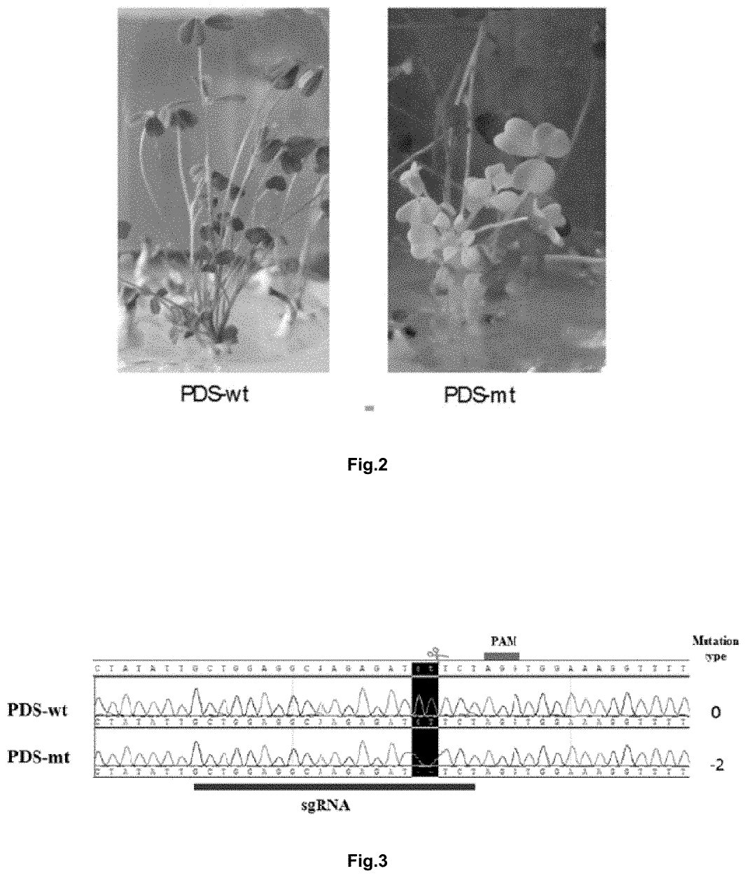

According to the PDS sequence in Medicago truncatula (a proximal species of Medicago sativa), a primer set was designed to amplify a PDS gene sequence of Medicago sativa and the PDS gene was sequenced; according to the resulting PDS gene sequence and with reference to the case in Medicago truncatula, a target site was designed; the DNA fragment containing the guide sequence of the target site was ligated into a site of the vector MsCRISPR/Cas9 to generate a vector for knocking out the PDS gene; the vector was transformed into the explant of Medicago sativa in the above transforming protocol by Agrobacterium tumefaciens, generating the regenerated plants (FIG. 2). Wherein, the construction of the expression vector, the preparation of Agrobacterium strains, the transformation by means of Agrobacterium, and the screening and genotyping of mutants were as below:

First. The expression vector was constructed as below:
1. Designing a pair of oligonucleotide primers (18-24 bp) according to the target site, which can form DNA fragments with cohesive ends at both ends as below, and synthesize:

5′-T T T G N₁₆₋₂₃-3′
3′-C N₁₆₋₂₃C A A A-5′;

2. Annealing the above synthesized primer pair;
3. Digesting the vector MsCRISPR/Cas9 with Aarl restriction enzyme; after reaction with CIP, separating in 0.8-1.2% of agarose gel, cutting the gel and purifying the linear vector;
4. Ligating the annealed and phosphorylated DNA fragments to the gel recycled MsCRISPR/Cas9 linear vector by using a T4 DNA ligase in a water-bath pot at 16° C. for 10-16 h;
5. Transforming Escherichia coli DH5α competent cells with the ligated products by heat shock, coating on a LB plate medium containing 50 mg/L of kanamycin, and cultivating in an incubator at 37° C. for 12-16 h;
6. Selecting monoclones in 1.5 ml LB liquid medium containing 50 mg/L of kanamycin, and culturing in a shaker at 37° C. for 12-16 h;
7. Sequencing the vector of monoclonal bacterials by using a sequencing primer, selecting proper monoclonal bacterials and cultivating them in 50-100 mL LB liquid medium containing 50 mg/L of kanamycin, culturing in a shaker at 37° C. for 12-16 h, and extracting the vectors.

The sequencing primer is as below:

MtU6-T-F:
GGCATGCAAGCTTATCGATAC.

Second. Preparation of Agrobacterium Strains

The above expression vector was transformed into Agrobacterium tumefaciens strain EHA105 competent cells by electroporation, recovered and coated in a YM medium (with 50 mg/L of kanamycin and 250 mg/L of rifampicin), cultured at 28° C. for 24-48 h; monoclones were selected and inoculated in 30-50 mL YM liquid medium (with 50 mg/L of kanamycin and 250 mg/L of rifampicin), shaken at 28° C. in a shaker at 200 r/min for 24-48 h.

Third. Transformation by Means of Agrobacterium

A DNA sequence for expressing the CRISPR/Cas9 system and the Hpt protein that is resistant to the plant screening agent hygromycin was integrated into the genome of Medicago sativa by means of transformation with Agrobacterium, so that the expression products of these elements functioned to generate positive plants with the target gene silenced through screening. Specific transforming operations were as below:

1. Germination of Medicago sativa seeds: Plump and well-colored Medicago sativa seeds were selected, soaked with 75% alcohol for 2 minutes, washed with sterile water for two times, 1 minute for each time; then soaked with 0.1% mercuric chloride and shaken by hands for 10 minutes, and washed with sterile water for five times. The sterilized seeds were spread out in a large sterile culture dish containing filter papers, air-dried, inoculated on a MS solid medium, and germinated in light in an incubator or in a culture room for 7-14 days;

2. Induction of callus: Cotyledons and hypocotyls of the germinated seedlings were cut into small pieces with a sterile scalpel and inoculated on a callus induction medium, and cultured in dark in an incubator or in a culture room at 25±1° C. for 3 days. The ingredients of the callus induction medium were: SH basal medium+2 mg/L 2,4-D+0.2 mg/L KT+0.3 mg/L casein hydrolysate+30 g/L sucrose+8 g/L agar;

3. Infection: 1-2 days in advance, the prepared Agrobacterium tumefaciens strains were inoculated in 50-100 mL YM liquid medium and cultured at 28° C. on a shaker at 200 r/min until the OD value 260/280 was between 0.5-0.8; the bacterial solution was transferred into a 50 mL sterile centrifuge tube, and centrifuged at 4° C. in a centrifuge at r/min for 12 minutes; the centrifuge tube was taken out, the supernatant was discarded, and a resuspension solution was added for resuspension until the OD value 260/280 was between 0.5-0.8; Corresponding volume of acetosyringone at 100 μmol/ml was added according to adding 100 μmol acetosyringone per liter; the resuspension solution was MS liquid medium (MS+30 g/L sucrose); the explants induced in step 2 were collected into a 100 ml sterile triangular flask with a breathable and plastic sealing membrane, into which was poured a suitable amount of the resuspended bacterial solution (as long as covering the materials); the triangular flask was sealed with its own sealing membrane, and evacuated in a vacuum pump to 0.5 kpa for totally 1 h, during which the flask was shaken gently every 15 min; the triangular flask was taken out and shaken at 28° C. on a shaker at 120 r/min for 1 h; all the bacterial solution was poured out, and the materials were spread out in a sterile culture dish containing filter papers to dry out;

4. Co-cultivation: The dried transformed materials were inoculated on a co-cultivation medium (spreading a piece of sterile filter paper in the medium); the ingredients of the co-cultivation medium were: MS basal medium+2 mg/L 2,4-D+0.2 mg/L KT+30 g/L sucrose+8 g/L agar+100 μmol/L acetosyringone. They were cultivated in dark in an incubator or in a culture room at 25±1° C. for 3 days.

5. Screening: The co-cultivated materials were inoculated on a screening medium; the ingredients of the screening medium were: SH basal medium+2 mg/L 2,4-D+0.2 mg/L KT+30 g/L sucrose+8 g/L agar+250 mg/L cefotaxime+250 mg/L carbenicillin+15 mg/L hygromycin; they were cultivated in light in an incubator or in a culture room at 25±1° C. for 30-60 days;

6. Differentiation: The recovered materials were transferred onto a differentiation medium; the ingredients of the differentiation medium were: UM basal medium+2 g/L casein hydrolysate+0.4 mg/L kinetin+30 g/L sucrose+8 g/L agar+250 mg/L cefotaxime+5 mg/L hygromycin;

7. Rooting: The differentiated sprouts of 1-3 cm were transferred onto a rooting medium; the ingredients of the rooting medium were: MS basal medium+1 mg/L indolebutyric acid+30 g/L sucrose+8 g/L agar+250 mg/L cefotaxime.

Fourth. Screening and Genotyping of Mutants

Genotyping by targeted deep sequencing: DNA samples were extracted from the regenerated plants generated above, and amplified PDS sequence flanking the target site with specific amplification primers. PCR products were ligated into a pMD19-T vector by TA cloning, the Escherichia coli strain DH5α was transformed and then a batch of monoclones were selected for targeted deep sequencing to analyze whether there were mutations on the target as well as the types of mutations.

We found through experiments and genotyping:

As can be seen by phenotypic observation, an albino seedling was generated (FIG. 2). DNA samples were extracted from the albino seedling and the PDS gene sequence was amplified. The sequencing results (FIG. 3) showed that: there was a 2 bp base deletion at the target site of the plant, and the base deletion site was the cleavage site of Cas9 protein.

FIG. 2 showed that: wild-type (PDS-wt); mutant-type (PDS-mt). Wild-type plants showed normal green phenotype; while mutant plants showed white phenotype. The results were consistent with the phenotype of plants generated by PDS gene knockout mediated by CRISPR/Cas9 in white poplar, apple and other species, indicating that the resulting albino seedling was probably caused by the PDS gene mutation mediated by the introduced CRISPR/Cas9 system.

FIG. 3 showed that: As can be seen from the sequencing results, compared with wild-type plants, there was a 2 bp base deletion at the target site of the PDS mutant plants, and the mutation was located at the cleavage site of Cas9 protein, indicating that the albino seedling of Medicago sativa in FIG. 2 is caused by the 2 bp base deletion occurring in the process of gene repairing resulting from the cleavage of PDS gene target site by CRISPR/Cas9.

### Example 2 PALM1 Gene Mutation in Medicago sativa

This example is different from example 1 in that:

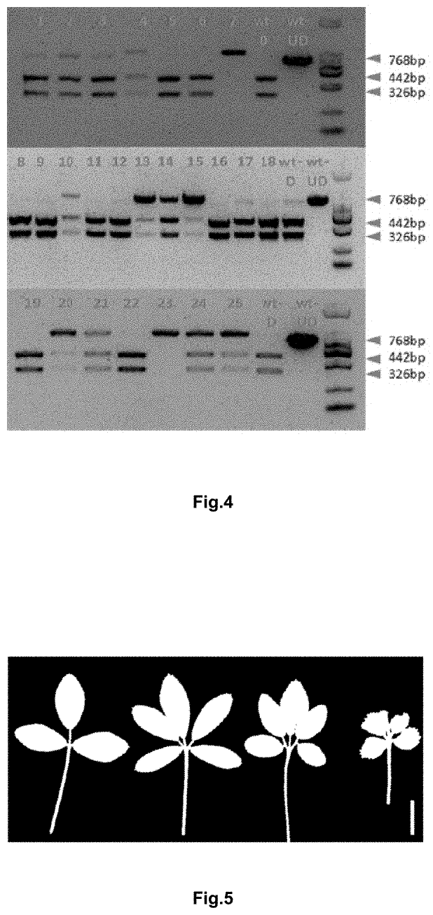

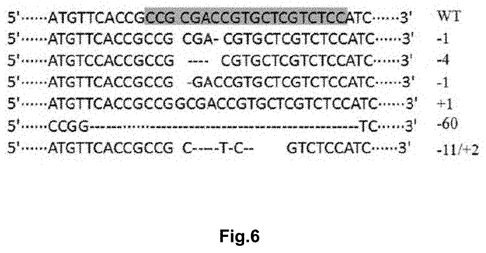

According to the PALM1 sequence in Medicago truncatula (a proximal species of Medicago sativa), a primer set was designed to amplify a MsPALM1 gene sequence of Medicago sativa and the MsPALM1 gene was sequenced; according to the resulting MsPALM1 gene sequence, a target site was designed; DNA fragment containing the guide sequence of the target site was ligated into the vector MsCRISPR/Cas9 to generate a vector MsCRISPR/Cas9::PALM1 for knocking out the MsPALM1 gene; the vector was transformed into the explant of Medicago sativa in the above transforming protocol by Agrobacterium tumefaciens, generating the regenerated plants. The genotyping method was PCR-RE, in which genome DNA were extracted from the regenerated plants generated above, and amplified fragment flanking the target site with specific amplification primers; and PCR products were digested with BstUI restriction endonuclease to screen mutant strains (FIG. 4); and the mutations were confirmed by phenotype observation (FIG. 5) and targeted deep sequencing (FIG. 6).

FIG. 4 showed that: As shown by PCR-RE genotyping, in the sampled 25 Medicago sativa regenerated plants, there were 13 mutant plants (there were bands that could not be digested with restriction endonuclease BstUI at 768 bp), wherein there were 10 heterozygous mutant plants (besides the bands that could not be digested with restriction endonuclease BstUI at 768 bp, there were also two bands generated after digestion with restriction endonuclease BstUI, which are located at 442 bp and 326 bp, respectively), and 3 null mutants (there were only the bands that could not be digested with restriction endonuclease BstUI at 768 bp); the mutation rate reached 52%, wherein the mutation rate of null mutants reached 12%.

FIG. 5 showed that, in the sampled 25 Medicago sativa regenerated plants, the compound leaves of three plants (No. 2, 3, 4 from left to right) showed obvious phenotypic changes. Compared with the control plant (No. 1 from left to right), the number of leaflets in the compound leaves of the mutant plants changed from three to five. Wherein, the mutant plant No. 3 (i.e., No. 20 in FIG. 4) showed weak bands that could be digested with BstUI restriction endonuclease in PCR-RE results, and this may be due to that the plants of TO generation were chimera.

FIG. 6 showed that, a part of plants screened from FIG. 4 were selected to subject to targeted deep sequencing for the MsPALM1 gene, and mutations have been successfully introduced into the target site of the MsPALM1 gene.

The above results showed that site-specific mutagenesis has been achieved successfully in Medicago sativa by the CRISPR/Cas9 technology.

What is claimed is:

1. A method for site-specific mutagenesis of Medicago sativa genes by using a CRISPR/Cas9 system, wherein, the method comprises the following steps:
Step (1) Constructing a universal binary expression vector MsCRISPR/Cas9 that can be used for transforming Medicago sativa by Agrobacterium tumefaciens;
Step (2) Designing a CRISPR/Cas9-based target site for a target gene in the Medicago sativa, and ligating the DNA fragment containing the guide sequence of the target site into the universal vector MsCRISPR/Cas9 to construct a vector MsCRISPR/Cas9::target;
Step (3) Transforming the Medicago sativa by the Agrobacterium tumefaciens, and introducing site-specific mutations into the target gene.

2. The method for site-specific mutagenesis of Medicago sativa genes by using a CRISPR/Cas9 system according to claim 1, wherein, the Medicago sativa materials comprise wild varieties, local varieties, bred varieties, and introduced varieties.

3. The method for site-specific mutagenesis of Medicago sativa genes by using a CRISPR/Cas9 system according to claim 1, wherein, the target gene comprises any biologically functional genes or DNA sequences in the Medicago sativa genome.

4. The method for site-specific mutagenesis of Medicago sativa genes by using a CRISPR/Cas9 system according to claim 1, wherein, the complete sequence of the binary expression vector MsCRISPR/Cas9 for expressing the CRISPR/Cas9 system in the Medicago sativa is as shown in SEQ ID NO. 1.

5. The method for site-specific mutagenesis of Medicago sativa genes by using a CRISPR/Cas9 system according to claim 1, wherein, the backbone vector of the binary expression vector MsCRISPR/Cas9 for expressing the CRISPR/Cas9 system in the Medicago sativa is a pCambia1300 vector; the pCambia1300 backbone vector contains a T-DNA region that is used to transform the plants; the T-DNA region comprises a left boundary repeat (LB repeat), a right boundary repeat (RB repeat) as well as a sequence between the left and right boundary sequences used for transformation; the sequence between the left and right boundary sequences of the T-DNA region used for transformation comprises a Hpt gene expression frame, a Cas9 gene expression frame, an sgRNA expression frame; the Hpt gene expression frame comprises a CaMV 35S promoter (Enhanced) sequence for driving the transcription of the Hpt gene, a CDS sequence for expressing the Hpt gene and a CaMV poly A terminator sequence for terminating the transcription of the Hpt gene; the Cas9 gene expression frame comprises a 2×CaMV 35S promoter sequence for driving the transcription of the Cas9 gene, a CDS sequence of the Cas9 gene, and a Nos terminator sequence for terminating the transcription of Cas9 gene; the sgRNA expression frame comprises a MtU6 promoter from Medicago truncatula for driving the transcription of the sgRNA sequence and a DNA sequence for expressing sgRNA.

6. The method for site-specific mutagenesis of Medicago sativa genes by using a CRISPR/Cas9 system according to claim 5, wherein, the sequence of the MtU6 promoter is as shown in SEQ ID NO. 2.

7. The method for site-specific mutagenesis of Medicago sativa genes by using a CRISPR/Cas9 system according to claim 5, wherein, the DNA sequence for expressing sgRNA is as shown in SEQ ID NO. 3, and the DNA sequence from positions 1 to 30 comprises two Aarl restriction endonuclease recognition sites, which are used to construct the expression vector of site-specific mutagenesis for the target gene.

8. The method for site-specific mutagenesis of Medicago sativa genes by using a CRISPR/Cas9 system according to claim 1, wherein, the expression vector MsCRISPR/Cas9::target is constructed as below:
Step (1) Designing a pair of oligonucleotide primers 18-24 bp according to the target site, which can form DNA fragments with cohesive ends at both ends as below, and synthesize:
5′-T T T G N₁₆₋₂₃-3′
3′-C N₁₆₋₂₃C A A A-5′;
Step (2) The above synthesized primer pair is annealed to form a double-stranded DNA fragment of complementary oligonucleotide dimers with cohesive ends;
Step (3) Digesting the binary expression vector MsCRISPR/Cas9 with Aarl restriction endonuclease; dephosphorylating with calf alkaline phosphase CIP and then separating by electrophoresis in 0.8-1.2% of agarose gel, purifying the linear vector from gel;
Step (4) Ligating the complementary double-stranded DNA fragment with cohesive ends after annealing and dephosphorylation to the purified MsCRISPR/Cas9 linear vector by using a T4 DNA ligase in a water-bath pot at 16° C. for 10-16 h;
Step (5) Transforming Escherichia coli DH5α competent cells with the ligated products by heat shock, coating on a LB plate medium containing 50 mg/L of kanamycin, and cultivating in an incubator at 37° C. for 12-16 h;
Step (6) Selecting single colonies and cultivating them in 1.5 mL LB liquid medium containing 50 mg/L of kanamycin, and culturing in a shaker at 37° C. for 12-16 h;
Step (7) Sequencing the vector of monoclonal bacterials by using a sequencing primer MtU6-T-F, selecting proper monoclonal bacterials and cultivating them in 50-100 mL LB liquid medium containing 50 mg/L of kanamycin, culturing in a shaker at 37° C. for 12-16 h, and extracting the vectors.

9. The method for site-specific mutagenesis of Medicago sativa genes by using a CRISPR/Cas9 system according to claim 6, wherein, the sequence of the sequencing primer MtU6-T-F is as shown in SEQ ID NO. 4.

10. The method for site-specific mutagenesis of Medicago sativa genes by using a CRISPR/Cas9 system according to claim 1, wherein, the Medicago sativa is transformed by using Agrobacterium tumefaciens to generate mutant plants through the following steps:
Step (1) Recovery of the strains of Agrobacterium tumefaciens: The Agrobacterium strains after being transformed by heat shock are coated in a YM solid medium, and cultured at 25-29° C. for 24-48 h; monoclones are selected, inoculated in 30-50 mL YM liquid medium, and shaken on a shaker at 100-220 r/min at 25-29° C. for 24-48 h; the YM solid medium and the YM liquid medium both comprise 30-60 mg/L of kanamycin and 200-450 mg/L of rifampicin;
Step (2) Acquisition of Medicago sativa callus: The Medicago sativa callus is generated as below:
Plump and well-colored Medicago sativa seeds are selected, soaked with 65-80% alcohol for 1-3 minutes, washed with sterile water for 2-4 times, 0.5-2 minutes for each time; then soaked with 0.05-0.15% mercuric chloride and shaken by hands for 6-12 minutes, and washed with sterile water for 3-5 times; then inoculated on a MS solid medium, and germinated in light in an incubator or in a culture room for 7-16 days;
Cotyledons and hypocotyls of the germinated seedlings are cut into small pieces and inoculated in a callus induction medium, and cultured in dark in an incubator or in a culture room at 25±1° C. for 2-4 days; The ingredients of the callus induction medium are: SH basal medium+1.0-3.0 mg/L 2,4-Difluorophenoxy acetic acid+0.1-0.3 mg/L kinetin+0.1-0.5 mg/L casein hydrolysate+15-40 g/L sucrose+5.8-9 g/L agar; Preferably, the ingredients of the callus induction medium are: SH basal medium+2 mg/L 2,4-Difluorophenoxy acetic acid+0.2 mg/L kinetin+0.3 mg/L casein hydrolysate+30 g/L sucrose+8 g/L agar;
Step (3) Infection of Medicago sativa callus with Agrobacterium tumefaciens: the Medicago sativa callus is transformed with Agrobacterium strains as below: 1-2 days in advance, the Agrobacterium tumefaciens strains recovered in step (1) are inoculated in 50-100 mL YM liquid medium and cultured at 25-29° C. on a shaker at 100-220 r/min until the OD value 260/280 is between 0.5-0.8; the bacterial solution is transferred into a 50 mL sterile centrifuge tube, and centrifuged at 0-8° C. in a centrifuge at 3000-4500 r/min for 10-15 minutes; the supernatant is discarded, and a resuspension solution is added for resuspension until the OD value 260/280 is between 0.5-0.8; acetosyringone at 50-150 μmol/L is added; the resuspension solution is MS basal medium+30 g/L sucrose; the callus induced in step (2) is collected into a sterile triangular flask with a breathable and plastic sealing membrane, into which is poured the resuspended Agrobacterium bacterial solution, sealed with its own sealing membrane, and evacuated in a vacuum pump to 0.5 kpa for totally 0.5-1.5 h; the triangular flask is taken out and shaken at 25-29° C. on a shaker at 100-150 r/min for 0.5-1.5 h; all the bacterial solution is poured out and dried in the air;
Step (4) Co-cultivation of the Medicago sativa callus with Agrobacterium tumefaciens: The co-cultivation process is as below: The dried transformed materials are inoculated in a co-cultivation medium (spreading a piece of sterilized filter paper in the medium), and cultivated in dark in an incubator at 25±1° C. for 2-5 days; the ingredients of the co-cultivation medium are: MS basal medium+1-3 mg/L 2,4-Difluorophenoxy acetic acid+0.1-0.3 mg/L kinetin+15-40 g/L sucrose+5.8-9 g/L agar+50-150 μmol/L acetosyringone; Preferably, the ingredients of the co-cultivation medium are: MS basal medium+2 mg/L 2,4-Difluorophenoxy acetic acid+0.2 mg/L kinetin+30 g/L sucrose+8 g/L agar+100 μmol/L acetosyringone;
Step (5) Screening cultivation of Medicago sativa callus: The process of screening cultivation is as below: The co-cultivated materials are inoculated in a screening medium, and cultivated in light in an incubator or a culture room at 25±1° C. for 30-60 days; the ingredients of the screening medium are: SH basal medium+1-3 mg/L 2,4-Difluorophenoxy acetic acid+0.1-0.3 mg/L kinetin+15-40 g/L sucrose+5.8-9 g/L agar+150-450 mg/L cefotaxime+150-450 mg/L carbenicillin+10-50 mg/L hygromycin; Preferably, the ingredients of the screening medium are: SH basal medium+2 mg/L 2,4-Difluorophenoxy acetic acid+0.2 mg/L kinetin+30 g/L sucrose+8 g/L agar+250 mg/L cefotaxime+250 mg/L carbenicillin+15 mg/L hygromycin;
Step (6) Differentiation cultivation of Medicago sativa callus: The materials generated after screening cultivation are transferred into a differentiation medium, and cultivated in light in an incubator or a culture room at 25±1° C. for 15-30 days; the ingredients of the differentiation medium are: UM basal medium+0.5-5 g/L casein hydrolysate+0.1-2 mg/L kinetin+15-40 g/L sucrose+5.8-9 g/L agar+150-450 mg/L cefotaxime+4-10 mg/L hygromycin; Preferably, the ingredients of the differentiation medium are: UM basal medium+2 g/L casein hydrolysate+0.4 mg/L kinetin+30 g/L sucrose+8 g/L agar+250 mg/L cefotaxime+5 mg/L hygromycin;
Step (7) Rooting cultivation of regenerated sprouts of Medicago sativa: The differentiated sprouts of 1-3 cm are transferred into a rooting medium; the ingredients of the medium are: MS basal medium+0.5-2 mg/L indolebutyric acid+15-40 g/L sucrose+5.8-9 g/L agar+150-450 mg/L cefotaxime; Preferably, the ingredients of the rooting medium are: MS basal medium+1 mg/L indolebutyric acid+30 g/L sucrose+8 g/L agar+250 mg/L cefotaxime;
Step (8) Screening and genotyping of Medicago sativa mutants: The methods for screening and genotyping the Medicago sativa mutants comprise PCR-RE genotyping, T7E1 enzyme digestion genotyping and targeted deep sequencing.

11. An application of the method for site-specific mutagenesis of Medicago sativa genes by using a CRISPR/Cas9 system according to claim 1 in Medicago sativa breeding and Medicago sativa breeding.

12. The method for site-specific mutagenesis of Medicago sativa genes by using a CRISPR/Cas9 system according to claim 2, wherein, the expression vector MsCRISPR/Cas9::target is constructed as below:
Step (1) Designing a pair of oligonucleotide primers 18-24 bp according to the target site, which can form DNA fragments with cohesive ends at both ends as below, and synthesize:
5′-T T T G N₁₆₋₂₃-3′
3′-C N₁₆₋₂₃C A A A-5′;
Step (2) The above synthesized primer pair is annealed to form a double-stranded DNA fragment of complementary oligonucleotide dimers with cohesive ends;
Step (3) Digesting the binary expression vector MsCRISPR/Cas9 with Aarl restriction endonuclease; dephosphorylating with calf alkaline phosphase CIP and then separating by electrophoresis in 0.8-1.2% of agarose gel, purifying the linear vector from gel;
Step (4) Ligating the complementary double-stranded DNA fragment with cohesive ends after annealing and dephosphorylation to the purified MsCRISPR/Cas9 linear vector by using a T4 DNA ligase in a water-bath pot at 16° C. for 10-16 h;
Step (5) Transforming Escherichia coli DH5α competent cells with the ligated products by heat shock, coating on a LB plate medium containing 50 mg/L of kanamycin, and cultivating in an incubator at 37° C. for 12-16 h;
Step (6) Selecting single colonies and cultivating them in 1.5 mL LB liquid medium containing 50 mg/L of kanamycin, and culturing in a shaker at 37° C. for 12-16 h;
Step (7) Sequencing the vector of monoclonal bacterials by using a sequencing primer MtU6-T-F, selecting proper monoclonal bacterials and cultivating them in 50-100 mL LB liquid medium containing 50 mg/L of kanamycin, culturing in a shaker at 37° C. for 12-16 h, and extracting the vectors.

13. The method for site-specific mutagenesis of Medicago sativa genes by using a CRISPR/Cas9 system according to claim 3, wherein, the expression vector MsCRISPR/Cas9::target is constructed as below:
Step (1) Designing a pair of oligonucleotide primers 18-24 bp according to the target site, which can form DNA fragments with cohesive ends at both ends as below, and synthesize:
5′-T T T G N₁₆₋₂₃-3′
3′-C N₁₆₋₂₃C A A A-5′;
Step (2) The above synthesized primer pair is annealed to form a double-stranded DNA fragment of complementary oligonucleotide dimers with cohesive ends;
Step (3) Digesting the binary expression vector MsCRISPR/Cas9 with Aarl restriction endonuclease; dephosphorylating with calf alkaline phosphase CIP and then separating by electrophoresis in 0.8-1.2% of agarose gel, purifying the linear vector from gel;
Step (4) Ligating the complementary double-stranded DNA fragment with cohesive ends after annealing and dephosphorylation to the purified MsCRISPR/Cas9 linear vector by using a T4 DNA ligase in a water-bath pot at 16° C. for 10-16 h;
Step (5) Transforming Escherichia coli DH5α competent cells with the ligated products by heat shock, coating on a LB plate medium containing 50 mg/L of kanamycin, and cultivating in an incubator at 37° C. for 12-16 h;
Step (6) Selecting single colonies and cultivating them in 1.5 mL LB liquid medium containing 50 mg/L of kanamycin, and culturing in a shaker at 37° C. for 12-16 h;
Step (7) Sequencing the vector of monoclonal bacterials by using a sequencing primer MtU6-T-F, selecting proper monoclonal bacterials and cultivating them in 50-100 mL LB liquid medium containing 50 mg/L of kanamycin, culturing in a shaker at 37° C. for 12-16 h, and extracting the vectors.

14. The method for site-specific mutagenesis of Medicago sativa genes by using a CRISPR/Cas9 system according to claim 4, wherein, the expression vector MsCRISPR/Cas9::target is constructed as below:
Step (1) Designing a pair of oligonucleotide primers 18-24 bp according to the target site, which can form DNA fragments with cohesive ends at both ends as below, and synthesize:
5′-T T T G N₁₆₋₂₃-3′
3′-C N₁₆₋₂₃C A A A-5′;
Step (2) The above synthesized primer pair is annealed to form a double-stranded DNA fragment of complementary oligonucleotide dimers with cohesive ends;
Step (3) Digesting the binary expression vector MsCRISPR/Cas9 with Aarl restriction endonuclease; dephosphorylating with calf alkaline phosphase CIP and then separating by electrophoresis in 0.8-1.2% of agarose gel, purifying the linear vector from gel;
Step (4) Ligating the complementary DNA double-stranded DNA fragment with cohesive ends after annealing and dephosphorylation to the purified MsCRISPR/Cas9 linear vector by using a T4 DNA ligase in a water-bath pot at 16° C. for 10-16 h;
Step (5) Transforming Escherichia coli DH5α competent cells with the ligated products by heat shock, coating on a LB plate medium containing 50 mg/L of kanamycin, and cultivating in an incubator at 37° C. for 12-16 h;
Step (6) Selecting single colonies and cultivating them in 1.5 mL LB liquid medium containing 50 mg/L of kanamycin, and culturing in a shaker at 37° C. for 12-16 h;
Step (7) Sequencing the vector of monoclonal bacterials by using a sequencing primer MtU6-T-F, selecting proper monoclonal bacterials and cultivating them in 50-100 mL LB liquid medium containing 50 mg/L of kanamycin, culturing in a shaker at 37° C. for 12-16 h, and extracting the vectors.

15. The method for site-specific mutagenesis of Medicago sativa genes by using a CRISPR/Cas9 system according to claim 5, wherein, the expression vector MsCRISPR/Cas9::target is constructed as below:
Step (1) Designing a pair of oligonucleotide primers 18-24 bp according to the target site, which can form DNA fragments with cohesive ends at both ends as below, and synthesize:
5′-T T T G N₁₆₋₂₃-3′
3′-C N₁₆₋₂₃C A A A-5′;
Step (2) The above synthesized primer pair is annealed to form a double-stranded DNA fragment of complementary oligonucleotide dimers with cohesive ends;
Step (3) Digesting the binary expression vector MsCRISPR/Cas9 with Aarl restriction endonuclease; dephosphorylating with calf alkaline phosphase CIP and then separating by electrophoresis in 0.8-1.2% of agarose gel, purifying the linear vector from gel;
Step (4) Ligating the complementary double-stranded DNA fragment with cohesive ends after annealing and dephosphorylation to the purified MsCRISPR/Cas9 linear vector by using a T4 DNA ligase in a water-bath pot at 16° C. for 10-16 h;
Step (5) Transforming Escherichia coli DH5α competent cells with the ligated products by heat shock, coating on a LB plate medium containing 50 mg/L of kanamycin, and cultivating in an incubator at 37° C. for 12-16 h;
Step (6) Selecting single colonies and cultivating them in 1.5 mL LB liquid medium containing 50 mg/L of kanamycin, and culturing in a shaker at 37° C. for 12-16 h;
Step (7) Sequencing the vector of monoclonal bacterials by using a sequencing primer MtU6-T-F, selecting proper monoclonal bacterials and cultivating them in 50-100 mL LB liquid medium containing 50 mg/L of kanamycin, culturing in a shaker at 37° C. for 12-16 h, and extracting the vectors.

16. The method for site-specific mutagenesis of Medicago sativa genes by using a CRISPR/Cas9 system according to claim 6, wherein, the expression vector MsCRISPR/Cas9::target is constructed as below:
Step (1) Designing a pair of oligonucleotide primers 18-24 bp according to the target site, which can form DNA fragments with cohesive ends at both ends as below, and synthesize:
5′-T T T G N₁₆₋₂₃-3′
3′-C N₁₆₋₂₃C A A A-5′;
Step (2) The above synthesized primer pair is annealed to form a double-stranded DNA fragment of complementary oligonucleotide dimers with cohesive ends;
Step (3) Digesting the binary expression vector MsCRISPR/Cas9 with Aarl restriction endonuclease; dephosphorylating with calf alkaline phosphase CIP and then separating by electrophoresis in 0.8-1.2% of agarose gel, purifying the linear vector from gel;
Step (4) Ligating the complementary double-stranded DNA fragment with cohesive ends after annealing and dephosphorylation to the purified MsCRISPR/Cas9 linear vector by using a T4 DNA ligase in a water-bath pot at 16° C. for 10-16 h;
Step (5) Transforming Escherichia coli DH5α competent cells with the ligated products by heat shock, coating on a LB plate medium containing 50 mg/L of kanamycin, and cultivating in an incubator at 37° C. for 12-16 h;
Step (6) Selecting single colonies and cultivating them in 1.5 mL LB liquid medium containing 50 mg/L of kanamycin, and culturing in a shaker at 37° C. for 12-16 h;
Step (7) Sequencing the vector of monoclonal bacterials by using a sequencing primer MtU6-T-F, selecting proper monoclonal bacterials and cultivating them in 50-100 mL LB liquid medium containing 50 mg/L of kanamycin, culturing in a shaker at 37° C. for 12-16 h, and extracting the vectors.

17. The method for site-specific mutagenesis of Medicago sativa genes by using a CRISPR/Cas9 system according to claim 7, wherein, the expression vector MsCRISPR/Cas9::target is constructed as below:
Step (1) Designing a pair of oligonucleotide primers 18-24 bp according to the target site, which can form DNA fragments with cohesive ends at both ends as below, and synthesize:
5′-T T T G N₁₆₋₂₃-3′
3′-C N₁₆₋₂₃C A A A-5′;
Step (2) The above synthesized primer pair is annealed to form a double-stranded DNA fragment of complementary oligonucleotide dimers with cohesive ends;
Step (3) Digesting the binary expression vector MsCRISPR/Cas9 with Aarl restriction endonuclease; dephosphorylating with calf alkaline phosphase CIP and then separating by electrophoresis in 0.8-1.2% of agarose gel, purifying the linear vector from gel;
Step (4) Ligating the complementary double-stranded DNA fragment with cohesive ends after annealing and dephosphorylation to the purified MsCRISPR/Cas9 linear vector by using a T4 DNA ligase in a water-bath pot at 16° C. for 10-16 h;
Step (5) Transforming Escherichia coli DH5α competent cells with the ligated products by heat shock, coating on a LB plate medium containing 50 mg/L of kanamycin, and cultivating in an incubator at 37° C. for 12-16 h;
Step (6) Selecting single colonies and cultivating them in 1.5 mL LB liquid medium containing 50 mg/L of kanamycin, and culturing in a shaker at 37° C. for 12-16 h;
Step (7) Sequencing the vector of monoclonal bacterials by using a sequencing primer MtU6-T-F, selecting proper monoclonal bacterials and cultivating them in 50-100 mL LB liquid medium containing 50 mg/L of kanamycin, culturing in a shaker at 37° C. for 12-16 h, and extracting the vectors.

18. An application of the method for site-specific mutagenesis of Medicago sativa genes by using a CRISPR/Cas9 system according to claim 2 in Medicago sativa breeding.

19. An application of the method for site-specific mutagenesis of Medicago sativa genes by using a CRISPR/Cas9 system according to claim 3 in Medicago sativa breeding.

20. An application of the method for site-specific mutagenesis of Medicago sativa genes by using a CRISPR/Cas9 system according to claim 4 in Medicago sativa breeding.


SEQUENCE LISTING (4 sequences)

SEQ ID NO: 1 - DNA, 14951 bp, artificial sequence
  binary expression vector MsCRISPR/Cas9
aacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctggtcacctgtaattcacacgtggtggtggtggtggtggctagcgttaacactagtcagatctaccatggtggactcctcttagaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctctatcgatcaatcaggatccttactttttcttttttgcctggccggcctttttcgtggccgccggccttttgtcgcctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccggttcttgtcgcttctggtcagcaccttgttgtcgatggagtcgtccttcagaaagctctgaggcacgatatggtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggtttccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatgtccaggccgatgctgtacttcttgtcggctgctgggactccgtggataccgaccttccgcttcttctttggggccatcttatcgtcatcgtctttgtaatcaatatcatgatccttgtagtctccgtcgtggtccttatagtccatctcgagtatcgttcgtaaatggtgaaaattttcagaaaattgcttttgctttaaaagaaatgatttaaattgctgcaatagaagtagaatgcttgattgcttgagattcgtttgttttgtatatgttgtgttgaggtcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatatcaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcatgcaagcttatcgataccgtcgacctgcagccaagcttatccaacatttcacttgagttaactcaatagcaagaataacgtccatagtttcagcattcaagcaaaacggccaagaaaatcagcttggtaatttcagtgagacctggactaccataagcagcaccgcctattacacttaatggggtaaagtaaaacgagccacatcacctccttgattttaaggagcatttgaaggagtataaaaagaatgtatgtaatgtaaggttgtgttgtgtcattcaagatagcaagacggaccaaagcttctatgtatctatctatgtctatgatatgatgattgtattgatttggtttgagtacagtgagggagagggaggaacttcttcacttgtttatttaacctgaaactcaactcaaatcactgagagtgaatgttgagaaataagtattatgttatgtttgctttgctattagtcccacatcgcttacatatacttcagttatattgtttatatagcctagacgaacagcagggtttggctcgcaggtgaacacaacacctgcacacgttttagagctagaaatagcaagttaaaataaggctagtccgttatcaacttgaaaaagtggcaccgagtcggtgctttttttttaagcttggcgcgccgggcccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccgggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaactgcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagcacttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcac

SEQ ID NO: 2 - DNA, 500 bp, artificial sequence
  MtU6 promoter
atccaacatttcacttgagttaactcaatagcaagaataacgtccatagtttcagcattcaagcaaaacggccaagaaaatcagcttggtaatttcagtgagacctggactaccataagcagcaccgcctattacacttaatggggtaaagtaaaacgagccacatcacctccttgattttaaggagcatttgaaggagtataaaaagaatgtatgtaatgtaaggttgtgttgtgtcattcaagatagcaagacggaccaaagcttctatgtatctatctatgtctatgatatgatgattgtattgatttggtttgagtacagtgagggagagggaggaacttcttcacttgtttatttaacctgaaactcaactcaaatcactgagagtgaatgttgagaaataagtattatgttatgtttgctttgctattagtcccacatcgcttacatatacttcagttatattgtttatatagcctagacgaacagcagggttt

SEQ ID NO: 3 - DNA, 115 bp, artificial sequence
  DNA sequence expressing sgRNA
ggctcgcaggtgaacacaacacctgcacacgttttagagctagaaatagcaagttaaaataaggctagtccgttatcaacttgaaaaagtggcaccgagtcggtgcttttttttt

SEQ ID NO: 4 - DNA, 21 bp, artificial sequence
  sequencing primer MtU6-T-F
ggcatgcaagcttatcgatac